(19) United States
(12) Patent Application Publication
(10) Pub. No.: US 2020/0206173 A1
(43) Pub. Date: Jul. 2, 2020

(54) A PERSONAL CARE COMPOSITION

(71) Applicant: Conopco, Inc., d/b/a UNILEVER, Englewood Cliffs, NJ (US)

(72) Inventors: Chenliang Guo, Shanghai (CN); Nan HUANG, Shanghai (CN); Sheila Alves ROCHA, Bogota, NJ (US)

(73) Assignee: Conopco, Inc., d/b/a UNILEVER, Englewood Cliffs, NJ (US)

(21) Appl. No.: 16/641,701

(22) PCT Filed: Aug. 17, 2018
(86) PCT No.: PCT/EP2018/072342

§ 371 Date: Feb. 25, 2020

(30) Foreign Application Priority Data
Aug. 30, 2017 (CN) PCT/CN2017/099620
Sep. 20, 2017 (EP) 17192031.7

(51) Int. Cl. A61K 31/202; A61K 31/232; A61K 31/455; A61K 8/36; A61K 8/37; A61K 8/67; A61K 9/00; A61K 9/107; A61K 8/04; A61K 8/06; A61P 17/00; A61Q 19/00

(52) U.S. Cl. CPC A61K 31/202; A61K 31/232; A61K 31/455; A61K 8/361; A61K 8/37; A61K 8/675; A61K 2800/59; A61K 9/107; A61K 8/042; A61K 8/06; A61P 17/00; A61Q 19/00; A61K 9/0014

(57) ABSTRACT

The invention relates to a personal care composition, more particularly to a composition for use to reduce or prevent inflammation on a topical surface of a human or animal body. The composition could be delivered in the form of a skin, scalp, hair or oral care product, more particularly a skin care product. The benefit is delivered via a combination of a polyunsaturated fatty acid (PUFA) or an ester thereof and a vitamin B3 compound wherein the mole ratio of the PUFA or ester thereof to the vitamin B3 compound is 1:15 to 1:500.

### FIELD OF THE INVENTION

The invention relates to a personal care composition, more particularly to a composition for application to a topical surface of a human or animal that prevents or reduces inflammation. The composition could be delivered in the form of a skin, scalp, hair or an oral care product, more particularly a skin care product.

### BACKGROUND OF THE INVENTION

Inflammation, a complicated biological host response to harmful stimuli, is a mechanism by which the host removes the stimuli and initiates the healing process for self-protection. The innate immune system for a host is the first line of defence against invading organisms in a non-specific manner. Dysregulated inflammation may cause various personal care problems including gingivitis/periodontitis (in the oral cavity), dandruff (on scalp/hair) and eczema/acnes (on skin). To assist the host organism (e.g. the human or animal) several anti-inflammatory agents either through topical application or through oral consumption have been developed and used to mitigate the above problems.

The skin experiences many conditions like aging, pigmentation, photo-damage, and skin irritation which are all accompanied by inflammation. Additionally, inflammation has been implicated in many problems like skin wrinkling, acne, age spots, eczema, dandruff, and hyper-pigmentation. These problems, in addition to causing discomfort, also leave a displeasing cosmetic appearance. Many of the above problems have been treated or alleviated by use of one or more of emollients, sunscreens, anti-aging actives, or anti-microbial actives. Anti-inflammatory actives are also often included.

Thus, inflammation is a process that is manifest on the topical surface of the human or animal body in one or all of the above described conditions. The present inventors have attempted to alleviate the symptoms of the above conditions by developing combination of actives that exhibit synergistic anti-inflammatory benefits. The present invention concerns identification of actives which could be derivatized from compounds found in extracts of natural materials. Natural materials from which many actives have been extracted include several spices, ginger, turmeric, tea, grape, tomato and a host of others. The present inventors have studied combination of materials that could potentially provide synergistic anti-inflammatory activity. After extensive experimentation, they found that certain specific polyunsaturated fatty acids (PUFAs) in combination with vitamin B3 compound exhibit such behaviour and this is observed only at certain specific mole ratio range of the compounds.

WO13057284 A1 (Dignity Sciences) discloses compositions comprising fatty acids, or derivatives thereof (e.g., C1-C4 esters) including, for example, DGLA, 15-OHEPA and/or 15-HETrE, used singly or in combination with anti-bacterial agents for the treatment of disease and/or disorders such as acne or atopic dermatitis.

JP5286845 A (Kose, 1993) relates to a cosmetic which is chemically stable, and has effects of inhibiting skin ageing and improving skin roughening and comprises (A) eicosapentaenoic acid (EPA) and/or docosahexaenoic acid (DHA) or fat and/or oil containing them and (B) at least one or more selected from Japanese green gentian extract, camphor, nicotinic acid, or its derivatives, garlic essence, cepharanthine and Guinea pepper extract.

US2013095178 A1 (Manku Mehar) discloses pharmaceutical compositions for treatment of cardiovascular diseases like hyperlipidemia, hypertriglyceridemia and hypercholesterolemia. The compositions comprise eicosapentaenoic acid or a pharmaceutically acceptable ester and niacin.

US2012264705 A1 (Manku Mehar) discloses fatty acids, for example, DGLA, 15-OHEPA and/or 15-HETrE, alone or in combination with one or more antibiotic or anti-fungal agents for the treatment of disease and/or disorders such as a skin or gingival infection by topically applying the composition to an area afflicted with contusions, wounds, burns, sores, ulcers, scrapes, incisions, lacerations, skin infection, gingivitis or periodontal disease.

WO2011148247 A1 (Kerecis EHF) discloses a stabilized formulation for skin care, wound care and/or other tissue healing applications and methods for making the same is described. The stabilized formulation stabilizes omega-3 polyunsaturated fatty acids and is constituted of the omega-3 polyunsaturated fatty acids in combination with tocopherol (Vitamin E), ascorbic acid (Vitamin C), herb extract, and a fat-soluble antioxidant.

US2015050342 A1 (Lowe Nicholas) discloses cosmetic compositions comprising chlorhexidine and niacinamide or niacin; and salicylic acid for use in treating and preventing disorders of the skin which are associated with bacterial infections. In particular, the topical compositions of the invention are suitable for use in treating and preventing acne vulgaris. The specific combination of components, each of which is present at sub-irritant levels, but which combine synergistically to provide efficacy against acne and other inflammatory skin disorders which cannot be obtained using conventional treatments at sub-irritant levels.

The above publications do not disclose that a combination of PUFA and a vitamin B3 compound when formulated at specific ratios in a topical composition gives synergistic anti-inflammatory benefit.

It is thus an object of the present invention to provide for a personal care composition where the actives exhibit synergistic anti-inflammatory behavior.

It is another object of the present invention where at least one of the actives can be derived from a compound extractable from a natural source.

### SUMMARY OF THE INVENTION

According to the first aspect of the present invention there is provided a personal care composition for use to reduce or prevent inflammation on a topical surface of a human or animal body comprising:
(i) a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride, dihomogamma-Linolenic Acid (DGLA), DGLA-ethyl ester, DGLA-triglyceride or combinations thereof;
(ii) a vitamin B3 compound; and
(iii) a cosmetically acceptable base;
wherein the mole ratio of the PUFA or ester thereof to the vitamin B3 compound is 1:15 to 1:500.

According to another aspect of the present invention there is provided a non-therapeutic method of reducing or preventing inflammation on a topical surface of a human or animal body comprising a step of applying a composition on to the desired surface, said composition comprising:
(i) a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride, dihomogamma-linolenic Acid (DGLA), DGLA-ethyl ester, DGLA-triglyceride or combinations thereof;
(ii) a vitamin B3 compound; and,
(iii) a cosmetically acceptable base;
   wherein the mole ratio of the PUFA or ester thereof to the vitamin B3 compound is 1:15 to 1:500.

Yet another aspect of the present invention relates to non-therapeutic use of a composition for improving a skin condition including skin aging, pigmentation, photo-damage and skin irritation and inflammation, said composition comprising:
(i) a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride, dihomogamma-linolenic Acid (DGLA), DGLA-ethyl ester, DGLA-triglyceride or combinations thereof;
(ii) a vitamin B3 compound; and,
(iii) a cosmetically acceptable base;
   wherein the mole ratio of the PUFA or ester thereof to the vitamin B3 compound is 1:15 to 1:500.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the final composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

By ‘A topical composition’ or a ‘skin care composition’ as used herein, is meant to include a composition for topical application to the skin of mammals, especially humans. Such a composition could be of the leave-on or of the wash-off/rinse-off type. By a leave-on composition is meant a composition that is applied to the desired skin surface and left on for a period (say from one minute to 24 hours) after which it may be wiped or rinsed off with water, usually during the regular course of personal washing. By a wash-off/rinse off composition is meant a composition that is applied to the desired skin surface for a shorter period say of the order of seconds or minutes and usually contains sufficient surfactants that aids in cleaning the surface which may be rinsed off with copious amounts of water. The composition may also be formulated into a product which is applied to a human body for improving the appearance, cleansing, odor control or general aesthetics. The composition of the present invention can be in the form of a liquid, lotion, cream, foam, scrub, gel or stick form and may be delivered through a roll-on device or using a propellant containing aerosol can. “Skin” as used herein is meant to include skin on any part of the body e.g., neck, chest, back, arms, underarms, hands, legs, buttocks and scalp. When the product is used for the underarms it is usually called a deodorant product or a deo product. A class of deodorant product is the so called anti-perspirant (AP) product which contains an AP active which when applied to the axilla of an individual delivers anti-perspirancy and deodorancy benefits.

By a ‘Hair Care Composition” as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and wash-off shampoos, conditioners, shower gels, or toilet bar. The composition of the present invention is preferably a wash-off composition, especially preferred being a shampoo or a conditioner and most preferably a shampoo.

“Water-insoluble”, as used herein, refers to the solubility of a material in water at 25° C. and atmospheric pressure being 0.1% by weight or less.

According to the first aspect of the invention there is provided a personal care composition comprising a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride, dihomogamma-Linolenic Acid (DGLA), DGLA-ethyl ester, DGLA-triglyceride or combinations thereof; a a vitamin B3 compound; and a cosmetically acceptable base; wherein the mole ratio of the PUFA or ester thereof to the vitamin B3 compound is 1:15 to 1:500

The PUFA or ester thereof are preferably included in 0.01 to 10%, more preferably from 0.1 to 5% by weight of the composition. The preferred polyunsaturated fatty acids for inclusion in the composition of the invention are DHA, EPA, DGLA or DGLA ethyl ester.

The personal care composition of the invention comprises a Vitamin B3 compound. Vitamin B3 compound maybe niacin, nicotinic acid or niacinamide, preferably niacinamide.

Vitamin B3 compound is present in 0.01 to 10%, preferably present in 0.01 to 5%, more preferably 0.1 to 3% by weight of the composition.

The mole ratio of the PUFA or ester thereof to the Vitamin B3 compound is from 1:15 to 1:500 preferably from 1:20 to 1:100 more preferably 1:50 to 1:100.

The composition of the invention is preferably in the form of a skin, scalp or hair care product. Th cosmetically acceptable base preferably comprises water, oil, surfactant, emulsion, gel or combinations thereof. The cosmetically acceptable base in such products generally comprises one or more of water, oil, and surfactant and is generally in the form of an emulsion, gel or combinations thereof.

The composition of the invention preferably is used for skin care. The cosmetically acceptable base in such cases may be a liquid or solid material. Typically, base is present in an amount ranging from 10 to 99.9%, more preferably from 20 to 95%, most preferably from 40 to 85% by total weight of the composition including all ranges subsumed therein. It is particularly preferred that the cosmetically acceptable carrier includes water. Water is preferably included in an amount from 30 to 90%, more preferably from 30 to 85%, most preferably from 30 to 80% by total weight of the sunscreen composition. Besides water, suitable carrier classes include silicones, polyhydric alcohols, hydrocarbons, triglycerides and thickening powders.

The skin care composition could be in any form including toners, lotions, creams, mousses, scrub, serum or gel that is suitable for topical application to the skin. The composition can be either a leave-on product such as skin lotions, creams, antiperspirants, deodorants, lipsticks, foundations, mascara, sunless tanners and sunscreen lotions or a rinse-off product such as shower gels and toilet bars. It is preferred that the composition is a skin lotion or a cream.

The composition may comprise an emollient oil that act as a co-solvent. Suitable emollient oils include, for example, ester of alkoxylated aromatic alcohol with fatty carboxylic acid, esters of polyglycols or diols with fatty carboxylic acid such as caprylic/capric triglyceride, ester of fatty alcohol and fatty acid, alkoxylated derivative of benzyl alcohol and mixtures thereof. Preferably the emollient oil is caprylic/capric triglyceride.

Typically, such compositions comprise co-solvent in an amount from 0.01 to 10%, more preferably from 0.1 to 8%, most preferably from 1 to 6%, based on the total weight of the sunscreen composition.

The composition may additionally comprise sunscreen agents such as inorganic sunscreens. For example, zinc oxide, titanium dioxide, iron oxide, silica such as fumed silica. The amount of such sunscreen agents is preferably incorporated from 0.1 to 5% by total weight of the sunscreen composition.

The composition of the invention may comprise a UV-A sunscreen agent selected from the group consisting of a dibenzoylmethane derivative, a triazine derivative, a benzophenone derivative and mixtures thereof. In a preferred embodiment, the UV-A sunscreen agent comprises or is a dibenzoylmethane derivative, for example, butyl methoxydibenzoylmethane (sold under the trade name Parsol 1789).

Typically, the composition of the present invention comprises from 0.1 to 15% by weight of the UV-A sunscreen agent, more preferably from 0.1 to 10%, most preferably from 1 to 5%, based on the total weight of the composition.

The composition of the invention may also comprise a UV-B sunscreen agent. Suitable UV-B sunscreen agent of the invention is selected from the group consisting of a benzophenone, an anthranilate, a salicylate, a cinnamate, a camphor, benzylidene malonate, a triazone, and derivatives thereof. In a preferred embodiment, the UV-B sunscreen agent comprises or is a cinnamate derivative, for example, ethylhexyl methoxycinnamate (sold under the trade name Parsol MCX).

Typically, the composition of the invention preferably comprises from 0.1 to 20% by weight of the UV-B sunscreen agent, more preferably from 0.5 to 18%, most preferably from 1 to 15%, based on the total weight of the composition.

A skin lightening agent may also be incorporated into the composition of the invention. Suitable skin lightening agents other than Vitamin B3 and its derivatives (e.g. niacin, nicotinic acid, niacinamide) are kojic acid, arbutin, tranexamic acid, placental extract, ascorbic acid and its derivatives (e.g. magnesium ascorbyl phosphate, sodium ascorbyl phosphate, ascorbyl glucoside, and ascorbyl tetraisopalmitates), aloe extract, ammonium lactate, azelaic acid, citrate esters, ellagic acid, glycolic acid, green tea extract, hydroquinone, lemon extract, linoleic acid, vitamins other than B3 like vitamin B6, vitamin B12, vitamin C, vitamin A, a dicarboxylic acid, resorcinol derivatives, hydroxycarboxylic acid like lactic acid and their salts (e.g. sodium lactate) or a mixture thereof. Typically, the skin lightening agent is present in an amount from 0.1 to 10%, more preferably from 0.2 to 5%, most preferably from 0.3 to 3% by total weight of the composition.

The composition of the invention may also comprise other ingredients which are common in the art to enhance physical properties and performance. Suitable ingredients include but are not limited to humectants, thickeners, opacifiers, binders, colorants and pigments, pH adjusting agents, preservatives, optics, perfumes, viscosity modifiers, biological additives, buffering agents, conditioners, natural extracts, essential oils and skin benefit agents including other anti-inflammatory agents, cooling agents, antiperspirant agents, anti-aging agents, anti-acne agents, anti-microbial agents and antioxidants.

When the personal care composition is delivered for oral care, it includes a cosmetically acceptable base which comprises an abrasive, a thickener, a humectant and an orally acceptable surfactant.

Preferably, the composition of the invention is a composition for use in hair care. It is especially useful for preventing or alleviating the symptoms of dandruff. One medium through which this may be delivered is that of a shampoo. The composition of the invention especially shampoos are formulated with an anionic surfactant e.g. an alkyl sulphate and/or ethoxylated alkyl sulfate surfactant. These anionic surfactants are preferably present at a level of from 1 to 20%, preferably 2 to 16%, furthermore preferably from 3 to 16% by weight of the composition. Preferred alkyl sulfates are C8-18 alky sulfates, more preferably C12-18 alkyl sulfates, preferably in the form of a salt with a solubilising cation such as sodium, potassium, ammonium or substituted ammonium.

Shampoo compositions preferably comprise one or more further anionic cleansing surfactants which are cosmetically acceptable and suitable for topical application to the hair.

The compositions of the invention preferably additionally comprises an amphoteric surfactant preferably a betaine surfactant, more preferably an alkyl amido propyl betaine surfactant for example cocoamidopropyl betaine. In a preferred embodiment, the composition comprises from 0.1 to 10 wt. %, preferably from 0.5 to 8 wt. %, more preferably from 1 to 5 wt. % of a betaine surfactant

To enhance deposition of actives from compositions of the invention especially shampoos, cationic polymers are generally included therein. In accordance with the present invention too, it is preferred that the composition of the invention additionally includes 0.01 to 2.0% of a cationic polymer. The cationic polymer is preferably guar hydroxypropyl trimonium chloride. Guar polymer predominantly contains galactomannan polymer chains. This polymer is available at various molecular weights and degree of cationic substitutions depending on how much guar is hydrolysed and cationised.

Preferably the compositions of the invention comprise 0.04 to 0.5 wt %, more preferably 0.08 to 0.25 wt % by weight of the composition of the cationic polymer.

When conditioning benefits are to be delivered through the composition of the invention the composition is called a hair conditioner. Typically, the most popular conditioning agents used in hair care compositions are water-insoluble oily materials such as mineral oils, naturally occurring oils such as triglycerides and silicone polymers. Conditioning benefit is achieved by the oily material being deposited onto the hair resulting in the formation of a film, which makes the hair easier to comb when wet and more manageable when dry. An especially useful conditioning agent is a silicone compound, preferably a non-volatile silicone compound. Advantageously compositions herein may include one or more silicones. The silicones are conditioning agents found in dispersed or suspended particulate form. They are intended to deposit onto hair remaining behind after rinsing of the hair with water. Suitable silicone oils may include polyalkyl siloxanes, polyaryl siloxanes, polyalkylaryl siloxanes, polyether siloxane copolymers and mixtures thereof. Amino silicones are often formulated with shampoo compositions. Amino silicones are silicones containing at least one primary amine, secondary amine, tertiary amine or a quaternary ammonium group. High molecular weight silicone gums can also be utilized. Another useful type are the crosslinked silicone elastomers such as DimethiconeNinyl/Dimethicone Crosspolymers (e.g. Dow Corning 9040 and 9041).

Amounts of the silicone in compositions where present may range from about 0.1 to about 10 wt. %, preferably from about 0.1 to about 8wt. %, more preferably from about 0.3 to about 5wt. % by weight of the hair care compositions.

The pH of the composition is preferably equal to or higher than 4.0, more preferably in the range of 5.0 to 7.0.

The hair conditioning composition usually comprises conditioning surfactants selected from cationic surfactants, used singly or in admixture. Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arqued 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant. Yet another preferred cationic surfactant is stearamidopropyl dimethylamine.

The most preferred cationic surfactants for use in the composition are stearamidopropyl dimethylamine, behentrimonium chloride, or stearyl trimethyl ammonium chloride. In conditioners of the invention, the level of cationic surfactant will generally range from 0.1% to 5%, preferably 0.5 to 2.5% by weight of the composition.

Hair conditioning compositions of the invention preferably may also additionally comprise a fatty alcohol. The combined use of fatty alcohols and cationic surfactants in conditioning compositions is believed to be especially advantageous, because this leads to the formation of a lamellar phase, in which the cationic surfactant is dispersed.

The level of fatty alcohol in conditioners of the invention will generally range from 0.5 to 10%, preferably from 0.1% to 8%, more preferably from 0.2% to 7%, most preferably from 0.3% to 6% by weight of the composition

Hair care compositions whether delivered as shampoos or conditioners usually comprise an anti-dandruff agent. The most preferred anti-dandruff agent for use in the composition of the invention is a zinc based anti-dandruff agent preferably zinc pyrthione.

Shampoo composition as per the invention preferably additionally comprises a conazole fungicide. Preferably the conazole fungicide is selected form ketoconazole, climbazole or mixtures thereof. The azole fungicide is preferably included in 0.01 to 2%, more preferably 0.025 to 0.75% by weight of the composition. The presence of a conazole fungicide is believed to improve the deposition of zinc pyrithione.

The invention will now be illustrated with the help of the following non-limiting examples.

### EXAMPLES

### Examples 1 to 8: Synergistic Anti-Inflammation Efficacy of DHA with Niacinamide in THP-1 in Vitro Assay

The following procedure was used to test the anti-inflammation efficacy of the various actives alone or in combination.
(i) 5×10⁵ THP-1 monocytes were placed into 24-well plates, with 100 nM PMA in the culture media to differentiate the cell.
(ii) After 72 hours differentiation, treatments as follows were supplemented into fresh media and applied to the cells:
   Example 1: Vehicle control with LPS (1 μg/mL) stimulation
   Example 2,3: DHA at 100 or 20 μM alone with LPS (1 μg/mL) stimulation.
   Example 4,5: Niacinamide at 10000 or 5000 μM alone with LPS (1 μg/mL) stimulation.
   Example 6,7: Combinations of 100 μM DHA and either 10000 or 5000 μM of niacinamide.
   Example 8: Combination of 20 μM DHA and 10000 μM of niacinamide.
(iii) After 24 hours incubation, the supernatants were collected.
(iv) Then IL-6 concentration was quantified in the supernatant by BD Elisa kit.
(v) IL-6 inhibition rate was calculated in comparison with vehicle control with LPS stimulation as a reference for each treatment.

The results in terms of concentration of IL-6 as a % of that obtained for LPS is given in Table-1 below. The lower the percentage, the higher the anti-inflammatory potential. The CI value (combination index) of the various combinations is also less than 1 indicating synergism.

TABLE 1

| | | Mole ratio | | | |
|---|---|---|---|---|---|
| | | of DHA to | % of | Std. | CI |
| Examples | Composition | niacinamide | LPS | dev | value |
| 1 | LPS | — | 100 | | |
| 2 | 100 μM of DHA | — | 14.7 | 3.7 | |
| 3 | 20 μM of DHA | — | 58.5 | 3.2 | |
| 4 | 10000 μM of | — | 5.7 | 2.1 | |
| | niacinamide | | | | |
| 5 | 5000 μM of niacinamide | — | 33.4 | 5.5 | |
| 6 | 100 μM of DHA + 10000 | 1:100 | 2.1 | 1.0 | <1 |
| | μM of niacinamide | | | | |
| 7 | 100 μM of DHA + 5000 | 1:50 | 2.1 | 1.0 | <1 |
| | μM of niacinamide | | | | |
| 8 | 20 μM of DHA + 10000 | 1:500 | 3.6 | 0.4 | <1 |
| | μM of niacinamide | | | | |

The data in Table above indicates that a combination of DHA with niacinamide at various concentrations exhibits synergistic anti-inflammatory behaviour (with the CI value (combination index) of the various combinations is also less than 1 which indicated synergy between the polyunsaturated fatty acid and niacinamide.

### Examples 9 to 19: Anti-Inflammation Efficacy of Combination of DHA with Niacinamide at Various Other Concentrations

The data below is expressed in % IL-6 inhibition rate in Table-2 below.
(i) 5×10⁵ THP-1 monocytes were placed into 24-well plates, with 100 nM PMA in the culture media to differentiate the cell.
(ii) After 72 hours differentiation, treatments as follows were supplemented into fresh media and applied to the cells:
   Example 9: Vehicle control with LPS (1 μg/mL) stimulation
   Example 10,11,12: DHA at 100, 50 or 25 μM alone with LPS (1 μg/mL) stimulation.
   Example 13,14,15: Niacinamide at 5000, 2500 or 1250 μM alone with LPS (1 μg/mL) stimulation.
   Example 16: Combinations of 100 μM DHA and 5000 μM of niacinamide.
   Example 17: Combination of 50 μM DHA and 2500 μM of niacinamide.
   Example 18: Combination of 25 μM DHA and 1250 μM of niacinamide.
(iii) After 24 hours incubation, the supernatants were collected.
(iv) Then IL-6 concentration was tested in the supernatant by BD Elisa Kit.
(v) IL-6 inhibition rate was calculated in comparison with vehicle control with LPS stimulation as reference, for each treatment.

TABLE 2

| | | Mole ratio | | | |
|---|---|---|---|---|---|
| Example | | of DHA to | % of | Std. | CI |
| No. | Composition | niacinamide | LPS | dev | value |
| 9 | LPS | — | 100 | | |
| 10 | 100 μM of DHA | — | 8.3 | 0.6 | |
| 11 | 50 μM of DHA | — | 38.7 | 0.6 | |
| 12 | 25 μM of DHA | — | 65.5 | 2.5 | |
| 13 | 5000 μM of niacinamide | — | 26.5 | 0.4 | |
| 14 | 2500 μM of niacinamide | — | 50.8 | 1.6 | |
| 15 | 1250 μM of niacinamide | — | 64.5 | 3.3 | |
| 16 | 100 μM of DHA + 5000 | 1:50 | 1.4 | 0.4 | <1 |
| | μM of niacinamide | | | | |
| 17 | 50 μM of DHA + 2500 | 1:50 | 14.1 | 1.3 | <1 |
| | μM of niacinamide | | | | |
| 18 | 25 μM of DHA + 1250 | 1:50 | 35.4 | 0.7 | <1 |
| | μM of niacinamide | | | | |

The data in the table above indicates that at a mole ratio of 1:50 at various concentrations of the actives the combination shows synergy.

### Examples 19 to29: Anti-Inflammation Efficacy of Combination of DHA with Niacinamide at Mole Ratios Outside the Invention

Experiments were carried out at various concentrations of DHA and niacinamide at mole ratios outside the invention i.e., at 1:12.5. The data below is expressed in % IL-6 inhibition rate in Table-3 below:
(i) 5×10⁵ THP-1 monocytes were placed into 24-well plates, with 100nM PMA in the culture media to differentiate the cell.
(ii) After 72 hours differentiation, treatments as follows were supplemented into fresh media and applied to the cells:
   Example 20: Vehicle control with LPS (1 μg/mL) stimulation
   Example 21,22,23: DHA at 100, 50 or 25 μM alone with LPS (1 μg/mL) stimulation.
   Example 24,25,26: Niacinamide at 1250, 625 or 317.5 μM alone with LPS (1 μg/mL) stimulation.
   Example 27: Combinations of 100 μM DHA and 1250 μM of niacinamide.
   Example 28: Combination of 50 μM DHA and 625 μM of niacinamide.
   Example 29: Combination of 25 μM DHA and 317.5 μM of niacinamide.
(iii) After 24 hours incubation the supernatants were collected.
(iv) Then IL-6 concentration was tested in the supernatant by BD Elisa Kit.
(v) IL-6 inhibition rate was calculated in comparison with vehicle control with LPS stimulation as reference, for each treatment.

TABLE 3

| | | Mole ratio | | |
|---|---|---|---|---|
| Example | | of DHA to | % of | Std. |
| No. | Composition | niacinamide | LPS | dev |
| 20 | LPS | — | 100 | |
| 21 | 100 μM of DHA | — | 8.3 | 0.6 |
| 22 | 50 μM of DHA | — | 38.7 | 0.6 |
| 23 | 25 μM of DHA | — | 65.5 | 2.5 |
| 24 | 1250 μM of niacinamide | — | 35.4 | 0.7 |
| 25 | 625 μM of niacinamide | — | 77.6 | 0.9 |
| 26 | 317.5 μM of niacinamide | — | 84.2 | 2.2 |
| 27 | 100 μM of DHA + 1250 μM | 1:12.5 | 3.6 | 0.5 |
| | of niacinamide | | | |
| 28 | 50 μM of DHA + 625 μM of | 1:12.5 | 28.7 | 1.2 |
| | niacinamide | | | |
| 29 | 25 μM of DHA+ 317.5 μM | 1:50 | 52.7 | 1.8 |
| | of niacinamide | | | |

The data in Table-3 above indicates that at a mole ratio of 1:12.5, in most cases, synergy is not observed.

1. A personal care composition to reduce or prevent inflammation on a topical surface of a human or animal body, comprising:
(i) a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride or combinations thereof;
(ii) a vitamin B3 compound; and
(iii) a cosmetically acceptable base
wherein the mole ratio of the PUFA or ester thereof to the vitamin B3 compound is 1:20 to 1:100.

2. The composition as claimed in claim 1 wherein the PUFA or ester thereof is DHA or EPA.

3. The composition as claimed in claim 1 wherein the vitamin B3 compound is niacinamide.

4. The composition for as claimed in claim 1 comprising 0.01 to 10% PUFA or ester thereof by weight of the composition.

5. The composition as claimed in claim 1 comprising 0.01 to 10% vitamin B3 compound by weight of the composition.

6. The composition as claimed in claim 1 wherein the cosmetically acceptable base comprises water, oil, surfactant, emulsion, gel or combinations thereof.

7. The composition as claimed in any one of the preceding claim 1 in the form of an oral care, or a skin, scalp or hair care product.

8. A non-therapeutic method of reducing or preventing inflammation on a topical surface of a human or animal body comprising the step of applying a composition on to the surface, said composition comprising:
(i) a polyunsaturated fatty acid (PUFA) or an ester thereof selected from docosahexaenoic acid (DHA), eicosapentaenoic acid (EPA), DHA-ethyl ester, EPA-ethyl ester, DHA-triglyceride, EPA-triglyceride or combinations thereof;
(ii) a vitamin B3 compound; and,
(iii) a cosmetically acceptable base
wherein the mole ratio of the PUFA or ester thereof to the vitamin B3 compound is 1:20 to 1:100.

9. The non-therapeutic method of claim 8 wherein the composition further comprises a sunscreen.
